(19) United States
(12) Patent Application Publication
(10) Pub. No.: US 2025/0032522 A1
(43) Pub. Date: Jan. 30, 2025

(54) A Formulated And Synthesised Edible Birds Nest And Milk Based Nutritional Product And Methods For Preparation Thereof

(71) Applicant: Chee Boon MOH, BAYAN LEPAS (MY)

(72) Inventor: Chee Boon MOH, BAYAN LEPAS (MY)

(21) Appl. No.: 18/701,745

(22) PCT Filed: Jul. 30, 2023
(86) PCT No.: PCT/MY2023/050058

§ 371 Date: Apr. 16, 2024

(30) Foreign Application Priority Data
Aug. 22, 2022 (MY) PI2022004535

(51) Int. Cl. A61K 31/702; A23C 3/023; A23C 9/20; A61K 9/107; A61K 9/127; A61K 31/7008; A61K 35/57

(52) U.S. Cl. CPC A61K 31/702; A23C 3/023; A23C 9/203; A61K 9/1075; A61K 9/127; A61K 31/7008; A61K 35/57; A23C 2210/15; A23C 2210/40

(57) ABSTRACT

This invention relates to formulating, synthesising and producing an edible bird's nest and milk based nutritional product to enhance human brain health and immune function. The invention includes a plurality of methods in “synthesising” cleaned edible bird's nest, ultra-low fat milk, purified water and optional ingredients under a pressurised, heat-assisted hydrolysis and subsequent rapid cooling process to produce said nutritional product, which characterised by containing high concentration of lactose, protein, N-acetylglucosamine and oligosaccharide-bound sialic acid, which included sialyllactose, sialyllactosamine and their derivatives or intermediates.

### TECHNICAL FIELD

This invention relates to formulating and synthesising a nutritional product as well as methods of producing the nutritional product, and in particular, formulating and synthesising an edible bird's nest and milk based nutritional product with high concentration of N-acetylglucosamine and oligosaccharide-bound sialic acid, which include sialyllactose and sialyllactosamine to enhance human brain health and immune function.

### BACKGROUND ART

According to World Health Organization (WHO) human brain health is an emerging and growing concept that encompasses neural development, plasticity, functioning and recovery across the life course.

To date, infant's brain development and elderly's neurodegeneration related issues are the two most challenging human brain health issues. In addressing the said brain health issues, it is crucial to have the capability to formulate, synthesise and produce an effective nutritional product with natural and safe raw ingredients.

Additionally, in the quest to formulate, synthesise and produce the said nutritional product, it is important to understand the vital nutrients or bioactive compounds for brain development, cognition improvement and immune function enhancement. Besides, it is critical to identify and select the natural and safe raw ingredients with the said vital nutrients or bioactive compounds, as well as to optimum the processes in “synthesising” the ingredients and producing the said nutritional product.

Bing Wang reported infant's brain development is a complex process that involves the anatomic growth of the brain and many biochemical, physiological, and psychological processes. The rate of initial brain growth exceeds that of any other organ or body tissue, and by two years of age, the brain reaches about 80% of its adult's weight.

Babies are born with all their neurons already formed. However, synaptic connections between these neurons are in large part established and elaborated after birth. Important neuro-developmental process that occur throughout this critical period include synaptic networking, dendritic arborisation, and cell multiplication and migration. Generally, synapses are formed at very rapid rate during the early months of life, usually achieving maximum density between 6 to1 2 months after birth.

Morgan B. and Winick M. reported high concentration of oligosaccharide-bound sialic acid in human milk during the first week of lactation is in coinciding with a time of rapid synthesis of brain sialoglycoproteins and gangliosides. In additional, Bing Wang reported pre-term human milk contained about 13-23% more oligosaccharide-bound sialic acid than full-term human milk during the first 3 months of lactation. Again, the human milk and biological data suggested that N-acetylglucosamine and oligosaccharide-bound sialic acid, included sialyllactose and sialyllactosamine are crucial nutrients for new-borns, especially the pre-term babies.

The American Academy of Pediatrics (AAP) and the World Health Organization (WHO) both recommend exclusive breastfeeding until an infant is at least 6 months of age. Bing Wang reported that dietary oligosaccharide-bound sialic acid supplementation improves learning and memory in piglets. In addition, Bing Wang also reported sialic acid can be synthesised into polysialic acid glycan (polySia), which associates with neural cell adhesion molecules (NCAM) to form polySia-NCAM. The polySia-NCAM impacts molecular interactions during synaptic plasticity and neural development.

Additionally, Paval D. reported there is evidence that polySia is effective in the regulation of molecules such as brain-derived neurotrophic factor (BDNF) and dopamine. It is known that both BDNF and dopamine can reciprocally potentiate each other and have effect on the pathogenesis of hyperactivity, stereotypes and autistic like behaviours. Al-Farsi and Shafai reported some studies have demonstrated a significant protective effect of breastfeeding on the development of autism spectrum disorder (ASD). In 2021, the Centers for Disease Control and Prevention (CDC) announced that 1 in 44 eight-year-old children were identified with autism. Between 30 to 50% percent of people with autism also have seizures or epilepsy. Studies found the brain ganglioside and glycoprotein sialic acid levels are higher in breastfed infants compared to formula-fed infants. This is due to formula milk contains limited oligosaccharide-bound sialic acid. Besides, according to C. Kunz the development of the intestinal flora can be stimulated by oligosaccharides containing N-acetylglucosamine that enhance the growth of Bifidobacterium bifidum and improve gut health.

Elderly's neurodegeneration related issues is another major issue of concern. According to Long and Holtzman, Alzheimer's Disease (AD) among elderly is a progressive neurodegenerative disorder that accounts for over 60% of 46.8 million cases of dementia reported in World Alzheimer Report 2016. Statistically, about 1 in 10 of people age 65 or older have symptom of AD. About 1 in 3 of people age 85 or older have AD. There are over 10 million new cases of dementia each year worldwide. Sundaram reported that the nerve cell death causes memory loss and personality changes and disrupts one's ability to carry out daily activities.

The highest levels of sialic acids are found in the brain, where they are expressed mainly in gangliosides and polySia-NCAM. Age-associated loss of sialic acid in the brain has been demonstrated to negatively affect the degenerative potential of neuronal fibers, neural plasticity and microglial phagocytosis. In addition, a decrease in ganglioside levels has been linked to increased neuronal loss in aged brains. Additionally, sialic acid has also been indicated to play important roles in AD pathogenesis. While gangliosides primarily affect amyloid beta (AB) accumulation and deposition, polySia-NCAM deficiency has been associated with reduced brain repair capabilities in AD. Again, the existing studies on AD indicating sialic acid is crucial for brain health enhancement and cognition improvement.

Therefore, it is an important objective of this invention to formulate, synthesise and produce a nutritional product, which is able to mimic human colostrum's oligosaccharides, containing high concentration of oligosaccharide-bound sialic acid and N-acetylglucosamine. The oligosaccharide-bound sialic acid included 3′-sialyllactose, 6′-sialyllactose, 3′-sialyllactosamine, 6′-sialyllactosamine and their derivatives or intermediates. Additionally, the said nutritional product is aims to provide the necessary monosaccharides and oligosaccharides “building block” for lactating mothers to biosynthesise their human milk with higher concentration of oligosaccharide-bound sialic acid, N-acetylglucosamine and other oligosaccharides.

Any discussion of documents, acts, materials, devices, articles or the like which has been included in the present specification is not to be taken as an admission that any or all of these matters form part of the prior art base or were common general knowledge in the field relevant to the present invention as it existed before the priority date of each claim of this application.

### DISCLOSURE OF INVENTION

The main objective of this invention is to formulate, synthesise and produce an effective nutritional product for human brain health and immune function enhancement, which mimic human colostrum of containing high concentration of oligosaccharide-bound sialic acid and N-acetylglucosamine. Additionally, the said nutritional product is particularly beneficial for elderly with neurodegenerative issue and women during their gestation and lactation period. The said nutritional product enable lactating mother to biosynthesise human milk with higher concentration of oligosaccharide-bound sialic acid and N-acetylglucosamine, which is crucial for infants' brain development, included memory and cognitive function improvement.

The present invention provides a formulated and synthesised nutritional product comprising of cleaned edible bird's nest, ultra-low fat milk, purified water and optional ingredients. The said nutritional product is characterised by containing high concentration of N-acetylglucosamine and oligosaccharide-bound sialic acid, included the 3′-sialyllactose, 6′-sialyllactose, 3′-sialyllactosamine, 6′-sialyllactosamine and their derivatives or intermediates.

The present invention includes a plurality of methods in “synthesising” the said cleaned edible bird's nest, said ultra-low fat milk, said purified water and said optional ingredients under a pressurised, heat-assisted hydrolysis process, and subsequent rapid cooling process to produce said nutritional product with high concentration of N-acetylglucosamine and oligosaccharide-bound sialic acid, which included the 3′-sialyllactose, 6′-sialyllactose, 3′-sialyllactosamine, 6′-sialyllactosamine and their derivatives or intermediates.

During the ingredients “synthesising” process, controlling and optimising process parameters (duration, pressure and temperature) of the said pressurised, heat-assisted hydrolysis process and the subsequent rapid cooling process, as well as the ingredients mixing weight ratio are crucial to enhance the N-acetylglucosamine concentration and the formation of oligosaccharide-bound sialic acid and their derivatives or intermediates from the original protein-bound sialic acid in edible bird's nest.

According to this invention, the said cleaned edible bird's nest, said ultra-low fat milk, said purified water and said optional ingredients are “synthesised” at a temperature above 90° C. for more than 10 minutes, preferable at a temperature in a range of 110° C. to 130° C. for more than 10 minutes to optimise release of said free and oligosaccharide-bound sialic acid from the said edible bird's nest. The said pressurised, heat-assisted hydrolysis process and the subsequent rapid cooling process are expected converting more than 15% of protein-bound sialic acid in the said edible bird's nest into said free and oligosaccharide-bound sialic acid, the preferable conversion rate is more than 50% of protein-bound sialic acid.

Besides, the said pressurised, heat-assisted hydrolysis process and the subsequent rapid cooling process also enhance “synthesise” of sialyllactose and sialyllactosamine from said sialic acid in edible bird's nest, and said lactose/N-acetyllactosamine in milk. Enzyme and/or mild acid are added as optional ingredients to further enhance or optimise the synthetisation of sialyllactose and sialyllactosamine.

Another objective of the invention is to provide a nutritional product with good sensory properties, which include appearance, texture, aroma and taste. In this invention, an ultra-low fat milk with high lactose and/or sialyllactose concentration is being used as one of the main ingredients. The ultra-low fat milk comprising of mammalian milk with less than 1.6% w/w of milk fat, preferable in a range of 0% to 0.1% w/w of milk fat in the milk liquid to minimise lipid-bound sialic acid formation, as well as to minimise chemical reaction between biomolecules of said milk fat and said edible bird's nest during the ingredients “synthesising” process, which affect the sensory, physical, chemical and/or biological properties of the said nutritional product.

Many nutritional products did not demonstrate their full potential of health benefits due to poor absorption rate in human body. Water solubility and low stability are mostly attributed to poor absorption in the human gastrointestinal (GI) tract. Orally administrated nutritional products must pass through the human GI tract and absorb by enteric epithelial cells. Therefore, it is another objective of the present invention to improve the absorption rate of the said nutritional product in the human gastrointestinal tract by introducing a homogenisation and encapsulation process, which is able to reduce the ingredients' particle size and increase the solubility, stability and bioavailability of the said nutritional product.

The above and other objects, features, aspects and advantages of the present invention will become clearer from the following detailed description of the present invention when taken in connection with the accompanying drawings. The drawings are for purpose of illustration only and not intended as a definition of the limits of the invention. It being understood that various changes in the details may be made without departing from the spirit and advantages of the present invention.

### BRIEF DESCRIPTION OF DRAWINGS

Embodiments of the invention are represented in the drawings and described in greater detail in the following description, in which drawings:

FIG. 1 shows chemical structure of sialic acid (N-acetylneuraminic acid, Neu5Ac or NANA), the sialic acid found in human brain, human milk and edible's bird nest.

FIG. 2 shows chemical structure of N-acetylglucosamine (Glc-NAc), the Glc-NAc found in human milk and edible bird's nest.

FIG. 3 shows chemical structure of glucose (Glc).

FIG. 4 shows chemical structure of galactose (Gal).

FIG. 5 shows chemical structure of fucose (Fuc).

FIG. 6 shows chemical structure of lactose, the lactose found in human milk and mammalian milk.

FIG. 7 shows chemical structure of N-acetyllactosamine (LacNAc), the LacNAc found in human milk and mammalian milk.

FIG. 8 shows chemical structure of lacto-N-biose (LNB), the LNB found in human milk and mammalian milk.

FIG. 9 shows changes in concentration of sialic acid (N-acetylneuraminic acid) in the full-term milk of eight mothers during the first 3 months of lactation (Janette Brand Miller, 1994).

FIG. 10 shows changes in concentration of N-acetylglucosamine (Glc-NAc) in the full-term milk of eight mothers during the first 3 months of lactation (Janette Brand Miller, 1994).

FIG. 11 shows changes in concentration of sialic acid (N-acetylneuraminic acid) in the full-term milk of a mother, who consumed the proposed nutritional product during the second and third month of lactation.

FIG. 12 shows changes in concentration of N-acetylglucosamine (Glc-NAc) in the full-term milk of a mother, who consumed the proposed nutritional product during the second and third month of lactation.

FIG. 13 shows chemical structures of 3′-sialyllactose found in human milk and the nutritional product according to the present invention.

FIG. 14 shows chemical structures of 6′-sialyllactose found in human milk and the nutritional product according to the present invention.

FIG. 15 shows chemical structures of 3′-sialyllactosamine found in human milk and the nutritional product according to the present invention.

FIG. 16 shows chemical structures of 6′-sialyllactosamine found in human milk and the nutritional product according to the present invention.

FIG. 17 shows a summary flow chart of a plurality of process combinations according to the present inventions.

FIG. 18 shows a process flow chart of producing a formulated and synthesised edible bird's nest and milk based nutritional product in accordance with example 1 of the present invention.

FIG. 19 shows a process flow chart of producing a formulated and synthesised edible bird's nest and milk based nutritional product in accordance with example 2 of the present invention.

FIG. 20 shows a process flow chart of producing a formulated and synthesised edible bird's nest and milk based nutritional product in accordance with example 3 of the present invention.

FIG. 21 shows a process flow chart of producing a formulated and synthesised edible bird's nest and milk based nutritional product in accordance with example 4 of the present invention.

FIG. 22 shows a process flow chart of producing a formulated and synthesised edible bird's nest and milk based nutritional product in accordance with example 5 of the present invention.

FIG. 23 shows a process flow chart of producing a formulated and synthesised edible bird's nest and milk based nutritional product in accordance with example 6 of the present invention.

FIG. 24 shows a process flow chart of producing a formulated and synthesised edible bird's nest and milk based nutritional product in accordance with example 7 of the present invention.

FIG. 25 shows a process flow chart of producing a formulated and synthesised edible bird's nest and milk based nutritional product in accordance with example 8 of the present invention.

FIG. 26 shows a process flow chart of producing a formulated and synthesised edible bird's nest and milk based nutritional product in accordance with example 9 of the present invention.

FIG. 27 shows a process flow chart of producing a formulated and synthesised edible bird's nest and milk based nutritional product in accordance with example 10 of the present invention.

FIG. 28 shows synthesis of 3′-sialyllactose in accordance with the present invention.

FIG. 29 shows synthesis of 6′-sialyllactose in accordance with the present invention.

FIG. 30 shows synthesis of 3′-sialyllactosamine in accordance with the present invention.

FIG. 31 shows synthesis of 6′-sialyllactosamine in accordance with the present invention.

FIG. 32 shows concentration of sialic acid in proposed nutritional product (mg/L) at different process temperature (°C) according to the present invention.

FIG. 33 shows a comparison of sialylated oligosaccharides, 3′-sialyllactose and 6′-sialyllactose in human milk and bovine milk (Sandra J M ten Bruggencate).

FIG. 34 shows a lipid bilayer encapsulation of a nutritional core material according to the present invention.

FIG. 35 shows a lipid monolayer encapsulation of a nutritional core material according to the present invention.

FIG. 36 shows a lipid encapsulation of a nutritional core material with combination of bilayer and monolayer according to the present invention.

FIG. 37 shows the minimum and preferred concentration of oligosaccharide-bound sialic acid, N-acetylglucosamine, lactose, protein and carbohydrate in the proposed nutritional product according to the present inventions.

FIG. 38 shows a plurality of examples of ingredients' weight ratio according to the present inventions.

### DETAIL DESCRIPTION OF INVENTION

The main objective of the present invention is to formulate, synthesise and produce an effective edible bird's nest and milk based nutritional product for human brain development, cognitive improvement and immune function enhancement, which mimic human milk oligosaccharides (HMOs) concentration, and in particular, human colostrum's sialylated oligosaccharides, sialic acid and N-acetylglucosamine concentration.

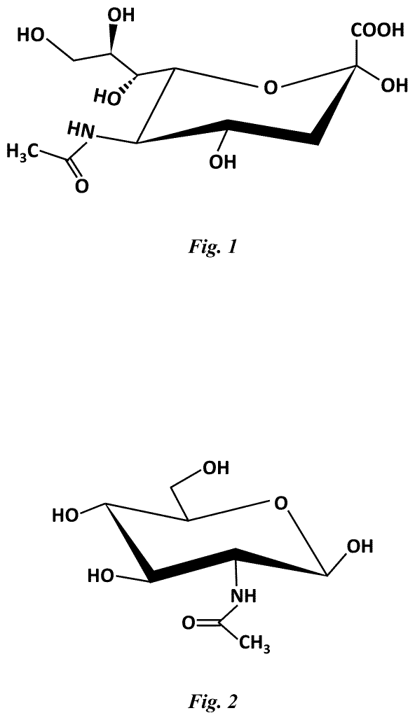

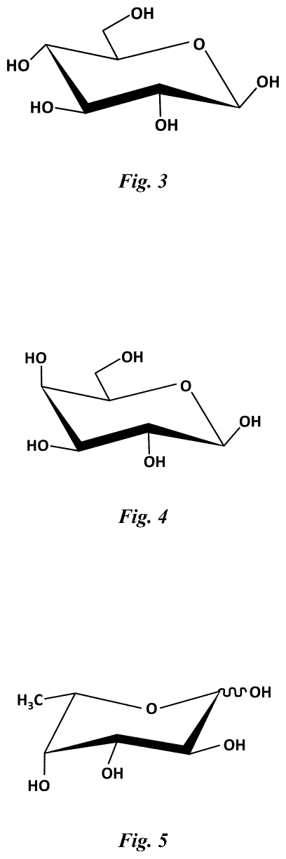

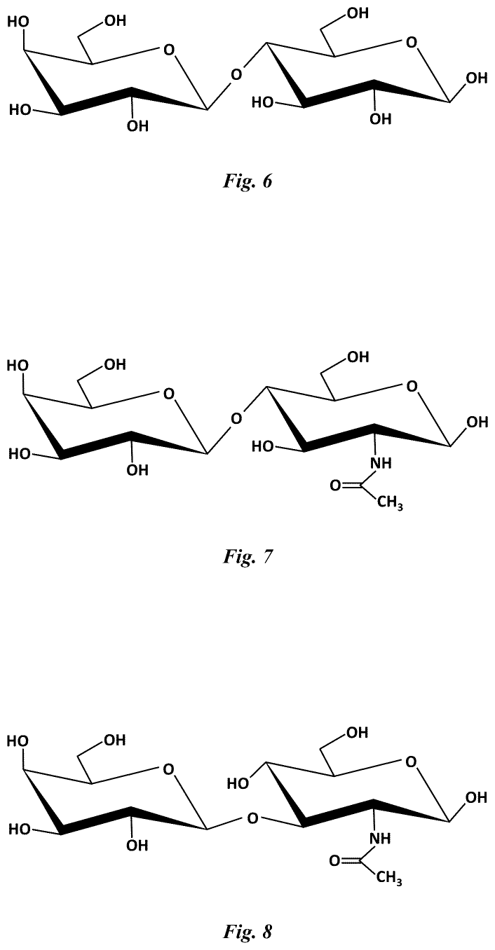

The human milk oligosaccharides (HMOs) are based on five monosaccharides residues, which are sialic acid (N-acetylneuraminic acid), N-acetylglucosamine (Glc-NAc), glucose (Glc), galactose (Gal) and fucose (Fuc). The chemical structures of sialic acid, Glc-NAc, Glc, Gal and Fuc are illustrated in FIG. 1, FIG. 2, FIG. 3, FIG. 4 and FIG. 5 respectively. Almost all HMOs contain lactose at the reducing end, which can be extended with N-acetyllactosamine or lacto-N-biose. The chemical structures of lactose, N-acetyllactosamine and lacto-N-biose found in human milk and mammalian milk are shown in FIG. 6, FIG. 7 and FIG. 8 respectively.

Human milk's oligosaccharide-bound sialic acid is an essential nutrient for brain development, cognitive improvement, immune system development and nervous system development. Oligosaccharide-bound sialic acid plays an important role in neurodevelopment of infants. In mammals, the highest concentration of sialic acid is in the central nervous system (CNS), mostly as structural and functional part in gangliosides and glycoproteins.

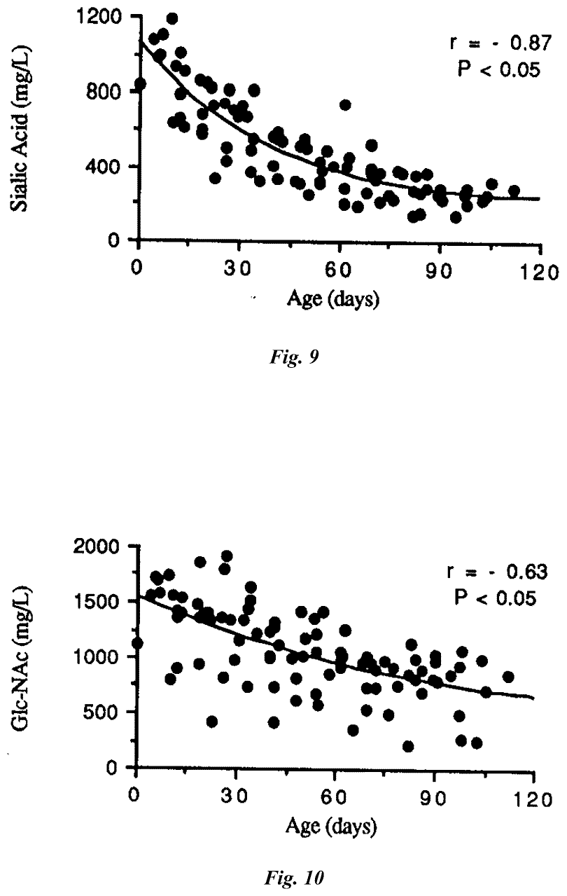

In human milk, sialic acid is present in different sialoglycoconjugate compounds such as oligosaccharides, glycolipids and glycoproteins. Human milk contains about 150-1200 mg/L of sialic acid during the first 3 months of lactation as shown in FIG. 9. Sialic acid bound to aligosaccharides accounts for about 75% of the total sialic acid contained in human milk. Most of the sialic acid contained in human milk is found in the form of sialyllactose (3′-sialyllactose and 6′-sialyllactose), which is an oligosaccharide-bound sialic acid formed from lactose and sialic acid. Some of the sialic acid in human milk is found in the form of sialyllactosamine (3′-sialyllactosamine and 6′-sialyllactosamine), which is an oligosaccharide-bound sialic acid formed from N-acetyllactosamine and sialic acid.

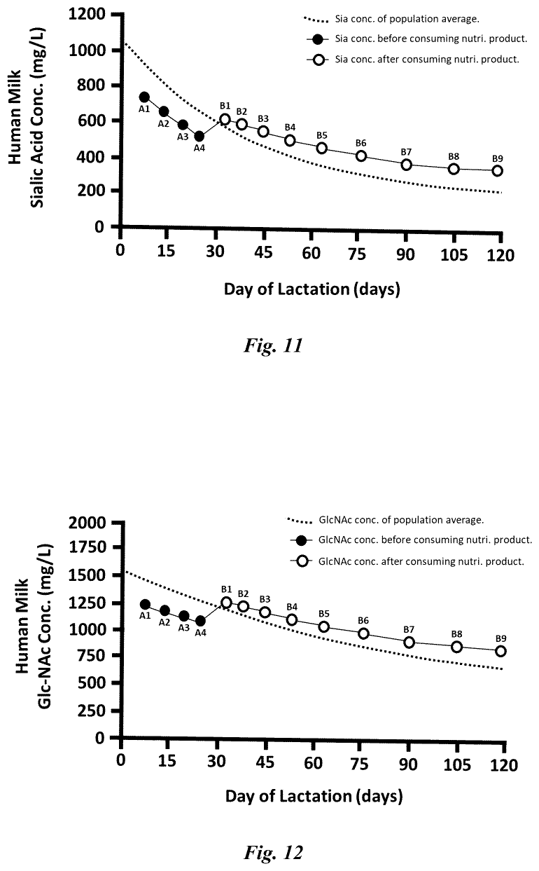

Human colostrum contains extremely high concentration of oligosaccharide-bound sialic acid (N-acetylneuraminic acid) and N-acetylglucosamine (Glc-NAc). The changes in concentration of sialic acid (N-acetylneuraminic acid) and N-acetylglucosamine (Glc-NAc) derived from the oligosaccharides in full term human milk of eight mothers during their first 3 months of lactation are shown in FIG. 9 and FIG. 10 respectively. As reported by Janette Brand Miller, sialic acid (N-acetylneuramic acid) showed an average 71% decrease in concentration over time, with the value fitting a quadratic curve over time (r=−0.87), declining from 879±157 mg/L (week 1) to 256±82 mg/L (week 13). Over the same period, the concentration of N-acetylglucosamine (Glc-NAc) declined by 56% from 1,459±282 mg/L (week1) to 646␣214 mg/L (week 13). The decreases in the concentration of sialic acid (N-acetylneuraminic acid) and N-acetylglucosamine (Glc-NAc) were both statistically significant (p<0.05). Naturally, the oligosaccharide-bound sialic acid and N-acetylglucosamine concentration in human milk are significantly higher at the beginning of lactation period, which match the new-borns' needs and their biological limitation in bio-synthesising the said compounds. The said high concentration of oligosaccharide-bound sialic acid and N-acetylglucosamine is crucial to new-borns biological system.

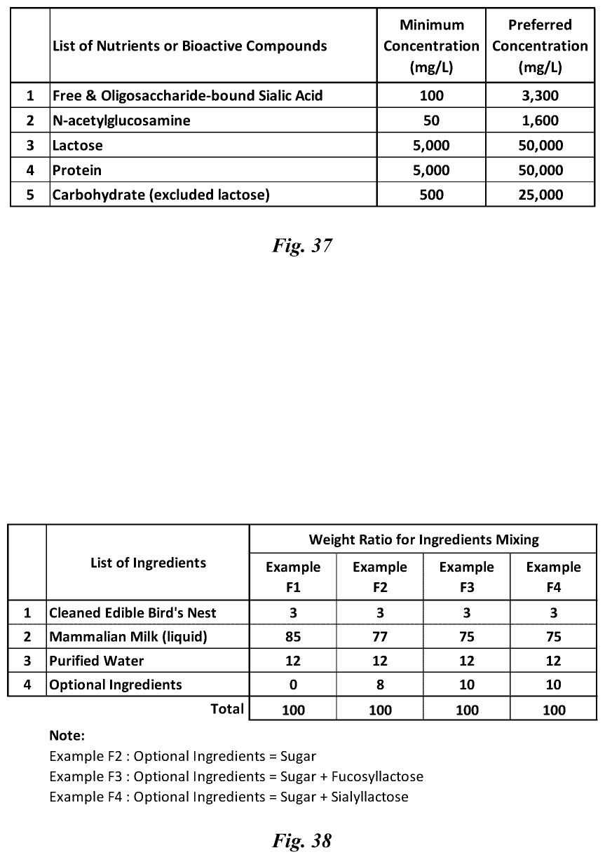

Therefore, it is an important objective of the present invention to mimic human colostrum in containing high concentration of oligosaccharide-bound sialic acid and N-acetylglucosamine. Additionally, the said nutritional product is particularly beneficial for elderly with neurodegenerative issue and women during their gestation and lactation period. The said nutritional product provides lactating mothers monosaccharides and oligosaccharides “building block”, especially the N-acetylglucosamine and oligosaccharide-bound sialic acid, which are the important components lactating mothers need to biosynthesise human milk, and to increase the concentration of oligosaccharide-bound sialic acid and N-acetlyglucosamine in their human milk. In this invention, the said nutritional product is characterised by containing high concentration of oligosaccharide-bound sialic acid, N-acetylglucosamine, lactose and protein, which concentrations are above 100 mg/L, 50 mg/L, 5000 mg/L and 5000 mg/L respectively. However, the preferred oligosaccharide-bound sialic, N-acetylglucosamine, lactose and protein concentration of the said nutritional product are at about 3,300 mg/L, 1,600 mg/L, 50,000 mg/L and 50,000 mg/L respectively, as shown in FIG. 37. FIG. 37 shown the minimum and preferred concentration of oligosaccharide-bound sialic acid, N-acetylglucosamine, lactose, protein and carbohydrate in the proposed nutritional product according to the present inventions.

According to the invention, a human trial was carried out on five lactating mothers of full-term babies to assess the effectiveness of the proposed nutritional product. The said lactating mothers were given the proposed nutritional product during their second and third month of lactation period. In this human trial, before and after the said lactating mothers were given the proposed nutritional product, their human milk samples were taken for oligosaccharide-bound sialic acid (N-acetylneuraminic acid) and N-acetylglucosamine concentration analysis.

The human trial data of one of the lactating mother is illustrated in FIG. 11 and FIG. 12. FIG. 11 shown the changes of sialic acid (N-acetylneuraminic acid) in the full-term milk of a mother, who orally administrated the proposed nutritional product during her second and third month of lactation. FIG. 12 shown the changes of N-acetylglucosamine (Glsc-NAc) in the full-term milk of a mother, who orally administrated the proposed nutritional product during her second and third month of lactation. By comparing the concentration of sialic acid (N-acetylneuraminic acid) and N-acetylglucosamine in the human milk before and after orally administrated the said nutritional product, as well as comparing the average concentration of sialic acid (N-acetylneuraminic acid) and N-acetylglucosamine in human milk of the control population, clearly observed both sialic acid and N-acetylglucosamine concentration in the human milk of the said lactating mother after orally administrated the said nutritional product is increased significantly and relatively higher and better than the average of control population.

The first 4 data points (A1, A2, A3 and A4) in FIG. 11 and FIG. 12 are the data points before oral administration of the said nutritional product. The subsequent 9 data points (B1, B2, B3, B4, B5, B6, B7, B8 and B9) in FIG. 11 and FIG. 12 are the data points after oral administration of the said nutritional product. The human trial data clearly concluded that the proposed nutritional product according to the invention significantly improved the concentration of sialic acid (N-acetylneuraminic acid) and N-acetylglucosamine in the human milk of the said lactating mother by 20-40% and 15-35% respectively. The changes of the concentration of sialic acid (N-acetylneuraminic acid) and N-acetylglucosamine in the human milk are largely depending on the doses orally administrated. Therefore, the said nutritional product formulation and the recommended oral administration doses are defined and decided based on the lactation period and the individual human milk's oligosaccharide concentration. Basically, the oligosaccharide-bound sialic acid (N-acetylneuraminic acid) and N-acetylglucosamine concentration in the human milk, as well as the lactation period are the two important indicators to decide the doses and frequency of the said nutritional product to be orally administrated.

Again, the above human trial data proven that oral administration of said nutritional product according to the invention with high concentration of oligosaccharide-bound sialic acid and N-acetlyglucosamine is crucial for lactating mother to improve her human milk's oligosaccharides concentration, as well as to benefit her baby indirectly via higher and better human milk oligosaccharides concentration.

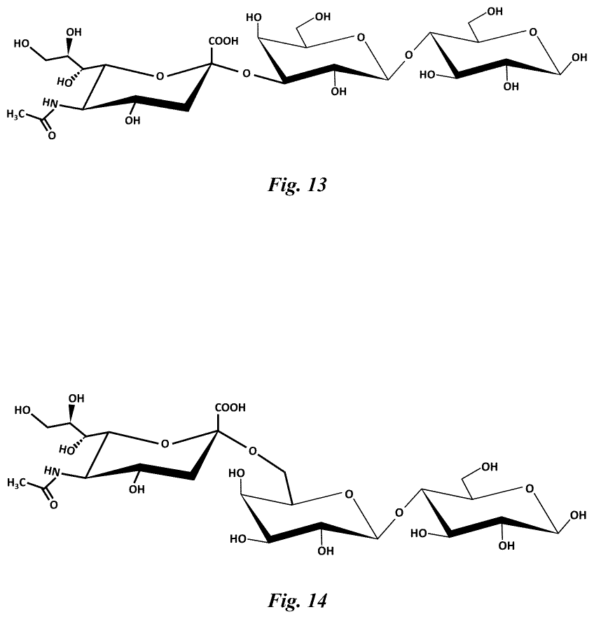

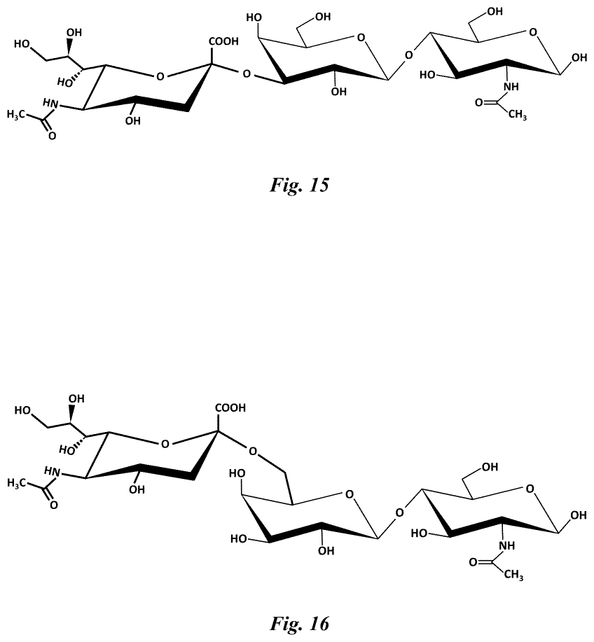

The present invention provides a formulated and synthesised nutritional product comprising of cleaned edible bird's nest, ultra-low fat milk, purified water and optional ingredients. The said nutritional product is characterised by containing high concentration of N-acetylglucosamine and oligosaccharide-bound sialic acid, included the 3′-sialyllactose, 6′-sialyllactose, 3′-sialyllactosamine, 6′-sialyllactosamine and their derivatives or intermediates. The chemical structures of the said 3′-sialyllactose, 6′-sialyllactose, 3′-sialyllactosamine and 6′-sialyllactosamine are illustrated in FIG. 13, FIG. 14, FIG. 15 and FIG. 16 respectively. In fact, similar chemical structures are found in both human milk and the proposed nutritional product according to the invention.

The term “Edible Bird's Nest” or EBN used in the description of this invention includes bird's nest made from regurgitated saliva of swiftlet. In particular, the EBN that built by Aerodramus Fuciphagus & Aerodramus Maximus swiftlets. The EBN chemical composition mainly consists of protein and carbohydrate, where 42-63% w/w is protein, 10.6-27.3% w/w is carbohydrate, 2.1-7.3% w/w is ash, and 0.1-1.3% w/w is lipid. The carbohydrate part of glycoprotein in EBN contains about 9-10% of sialic acid and about 5.3% of N-acetylglucosamine. Besides, the chemical structures of sialic acid monosaccharides and N-acetlyglucosamine monosaccharides found in raw EBN and human milk are the same, their chemical structures are shown in FIG. 1 and FIG. 2 respectively. The EBN chemical structures and chemical composition data clearly indicated that EBN is a good natural source of sialic acid (N-acetylneuraminic acid) and N-acetlyglucosamine to formulate and synthesise the said nutritional product. Therefore, EBN is selected as one of the main raw ingredients according to this invention.

Generally, the said raw EBNs are harvested from caves in South-East-Asia. The raw EBNs have to undergo a series of cleaning processes before they are ready to be cooked and consumed. Traditionally, the cleaned edible bird's nests are “cooked” or “double-boiled” with water and rock sugar. The “double-boiling” process is characterised by submerging a ceramic pot with an outer port of boiling water. Traditionally, the EBN is cooked or “double-boiled” at temperature about 90° C. for 30 minutes. At this cooking or processing setting, the raw EBN could not optimally releasing sialic acid monosaccharides from the original protein-bound sialic acid in the said EBN, as well as forming said oligosaccharide-bound sialic acid, included 3′-sialyllactose, 6′-sialyllactose, 3′-sialyllactosamine, 6′-sialyllactosamine and their derivatives or intermediates effectively. Therefore, majority of sialic acids found in the products prepared with the traditional method are not the oligosaccharide-bound sialic acids, they are mainly protein-bound sialic acids.

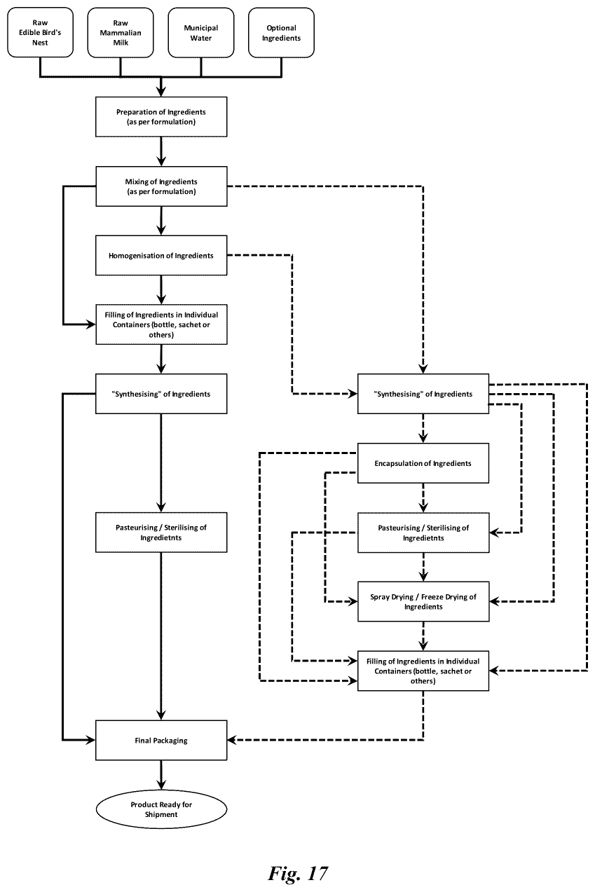

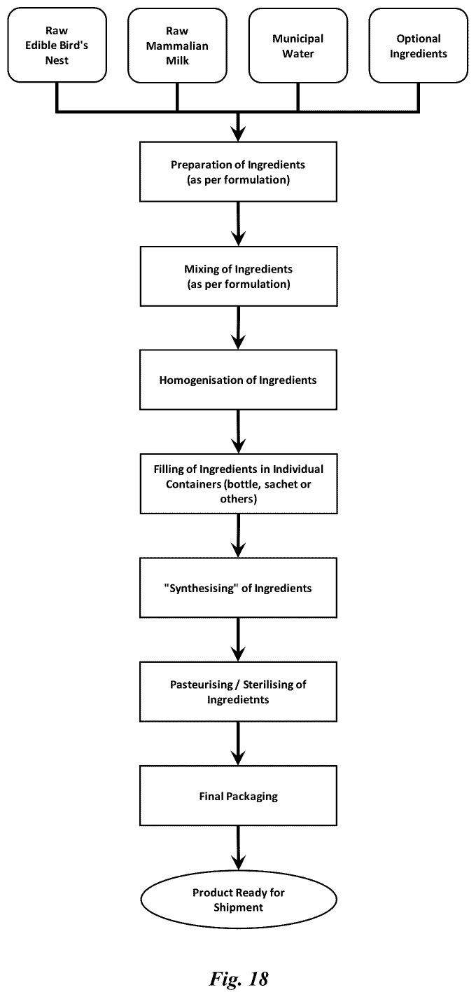

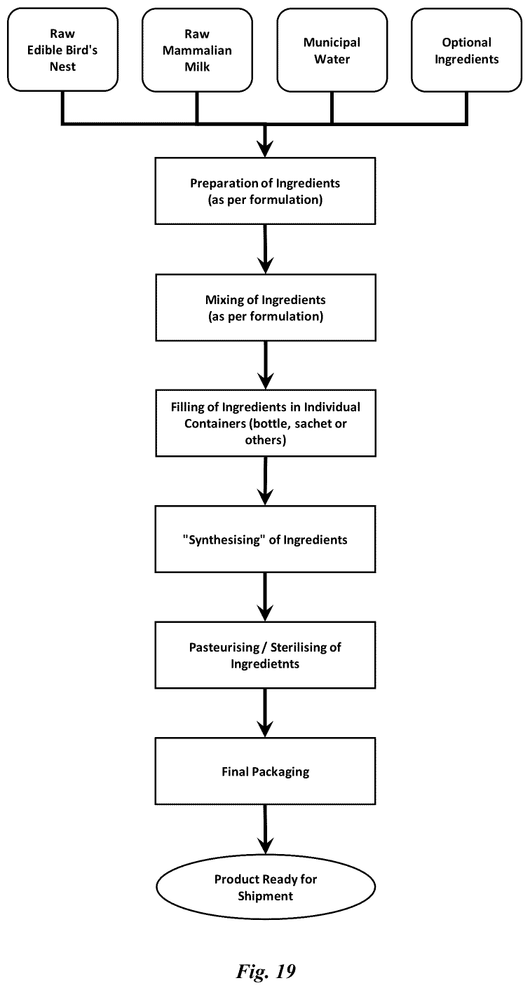

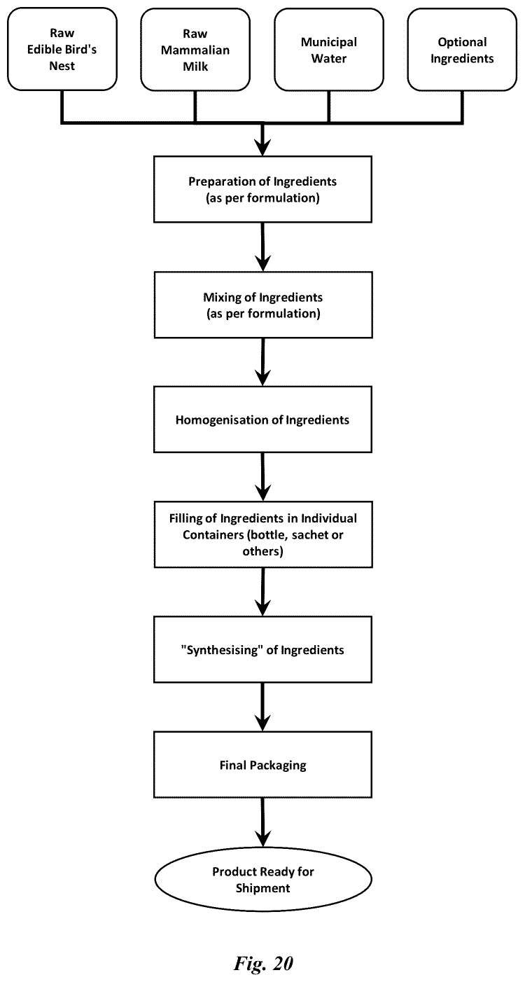

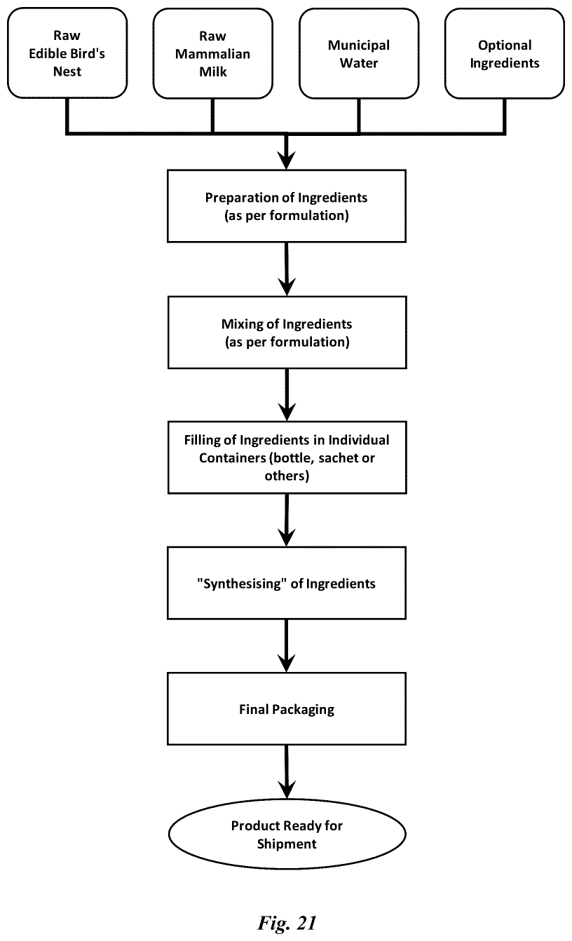

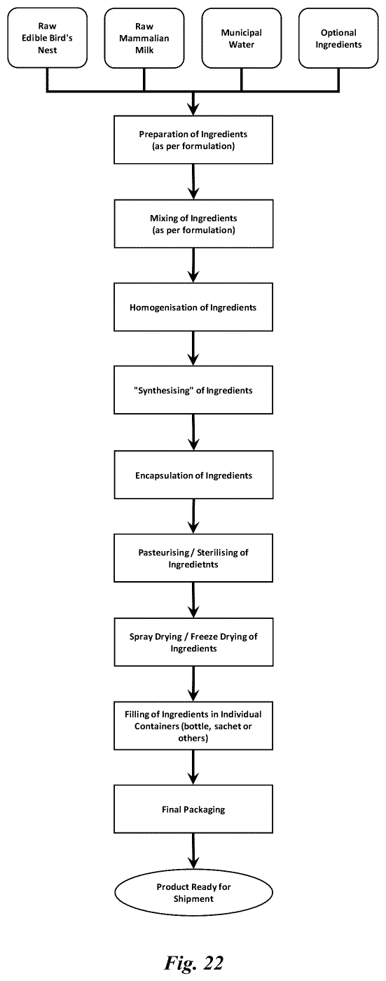

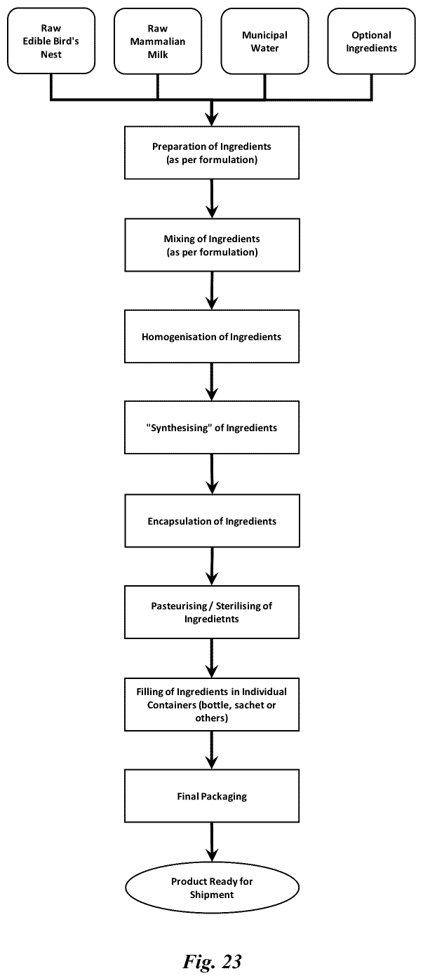

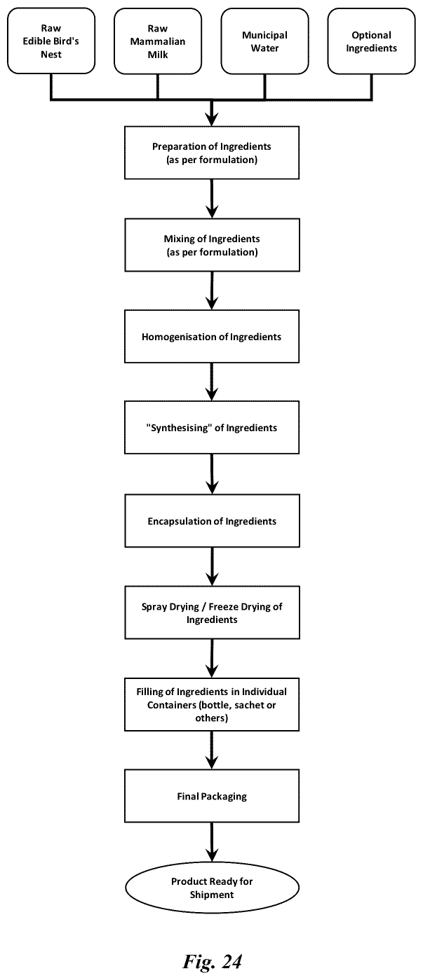

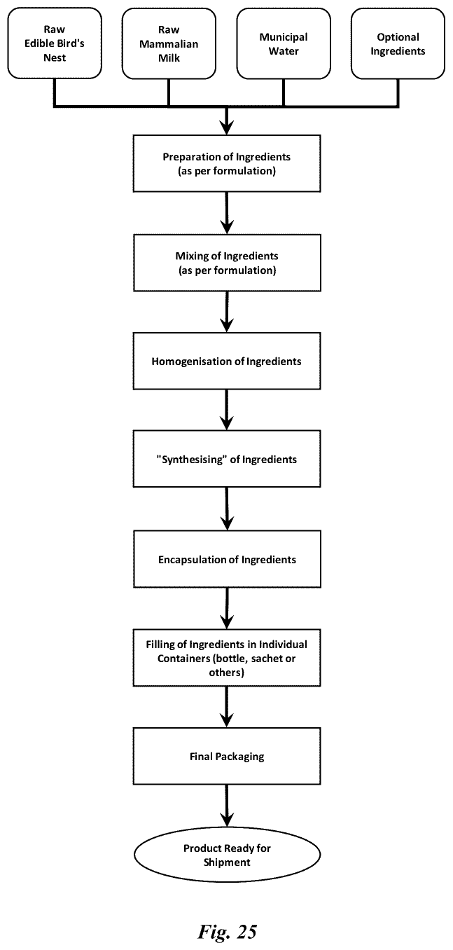

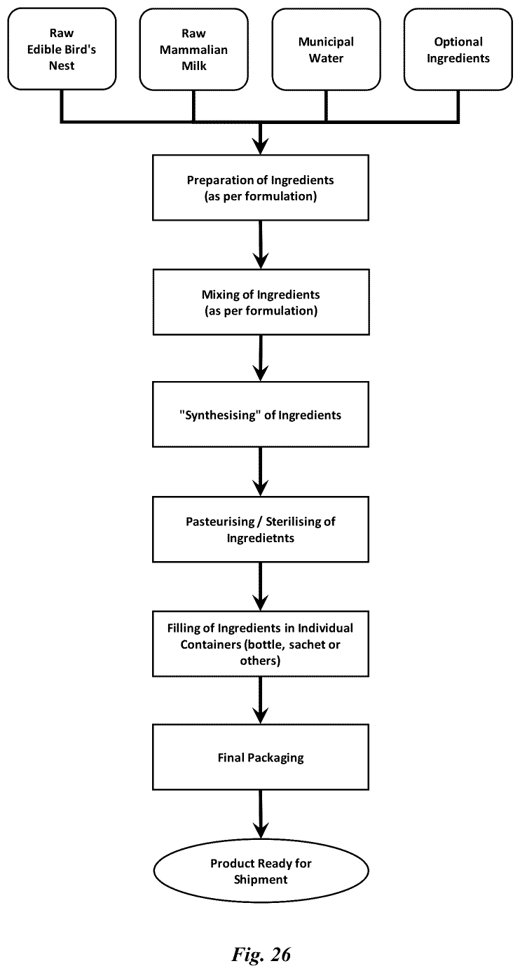

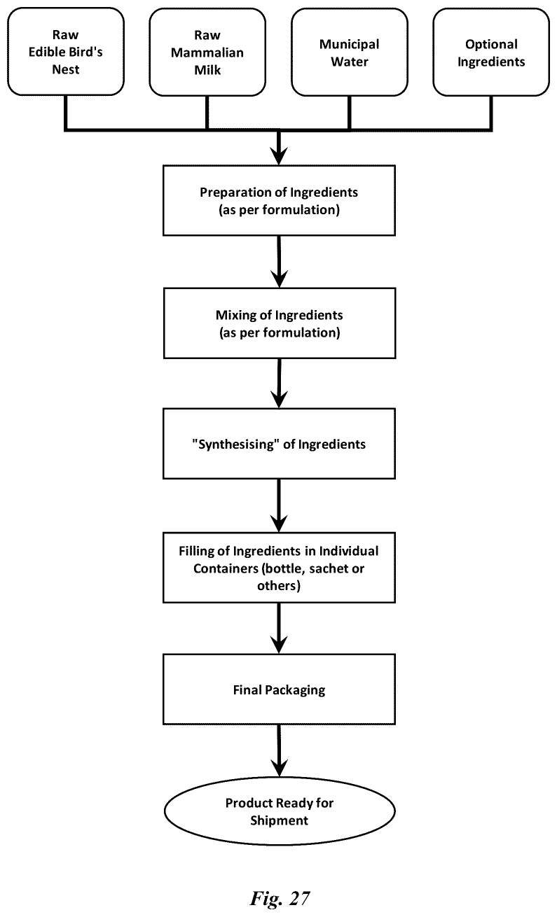

In this invention, a plurality of processes or methods being proposed to optimally and effectively produce the said nutritional product by enabling better releasing of sialic acids monosaccharides and also forming of said oligosaccharide-bound sialic acids, which eventually enable formation of more sialyllactose and sialyllactosamine and their derivatives or intermediates. FIG. 17 shown a summary flow chart of a plurality of process combinations according to the present invention. The plurality of methods to produce the said nutritional product is further illustrated in examples as shown FIG. 18, FIG. 19, FIG. 20, FIG. 21, FIG. 22, FIG. 23, FIG. 24, FIG. 25, FIG. 26 and FIG. 27.

The present invention proposed a plurality of unique methods to “synthesise” said cleaned edible bird's nest, said ultra-low fat milk, said purified water and said optional ingredients with a pressurised, heat-assisted hydrolysis process, and subsequent rapid cooling process to produce said nutritional product with high concentration of lactose, protein, N-acetylglucosamine and oligosaccharide-bound sialic acid, which included 3′-sialyllactose, 6′-sialyllactose, 3′-sialyllactosamine, 6′-sialyllactosamine and their derivatives or intermediates.

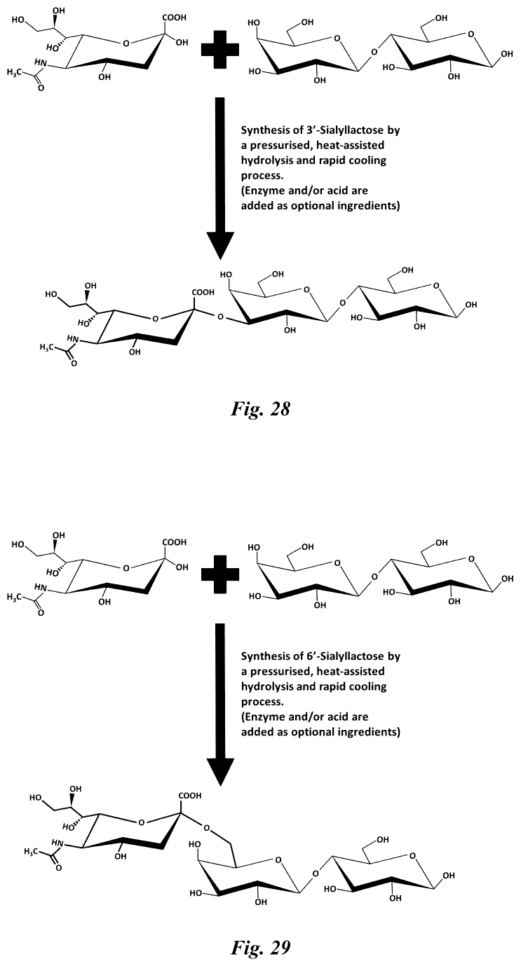

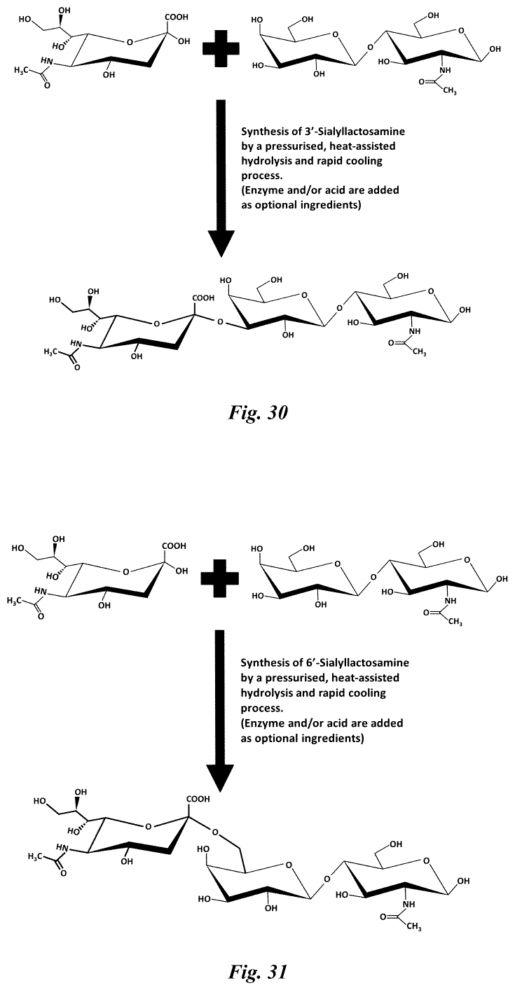

According to this invention, the said ingredients “synthesising” process is one of the important process, which means to increase the concentration of free and oligosaccharide-bound sialic acid, as well as the 3′-sialyllactose, 6′-sialyllactose, 3′-sialyllactosamine, 6′-sialyllactosamine and their derivatives or intermediates in the proposed nutritional product. In the present invention, the “synthesis” of 3′-sialyllactose, 6′-sialyllactose, 3′-sialyllactosamine and 6′-sialyllactosamine by the said pressurised, heat-assisted hydrolysis and the said subsequent rapid cooling process are further illustrated in FIG. 28, FIG. 29, FIG. 30 and FIG. 31 respectively. The said process enhanced synthesise or formation of sialyllactose and sialyllactosamine from said sialic acid in edible bird's nest, and said lactose/N-acetyllactosamine in milk. Enzyme and/or mild acid are added as optional ingredients to further enhance the “synthesising” process.

During the ingredients “synthesising” process, controlling and optimising the main process parameters (duration, pressure and temperature) of the said pressurised, heat-assisted hydrolysis process and the subsequent rapid cooling process, as well as the ingredients' weight ratio are crucial to determine the N-acetylglucosamine concentration and to enhance the formation of oligosaccharide-bound sialic acid and their derivatives or intermediates from the original protein-bound sialic acid in edible bird's nest and the said lactose/N-acetyllactosamine in milk. FIG. 38 shown a plurality of examples of ingredients' weight ratio according to the present inventions.

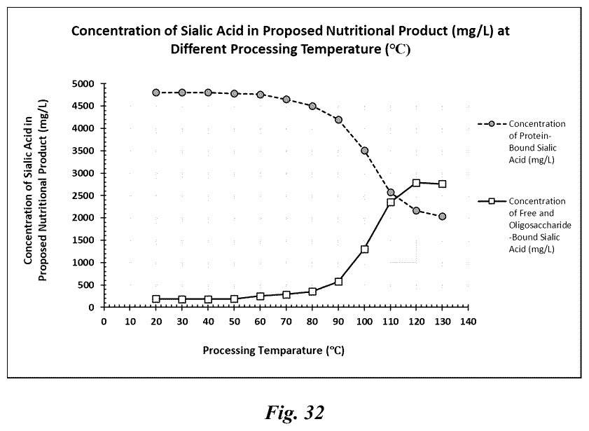

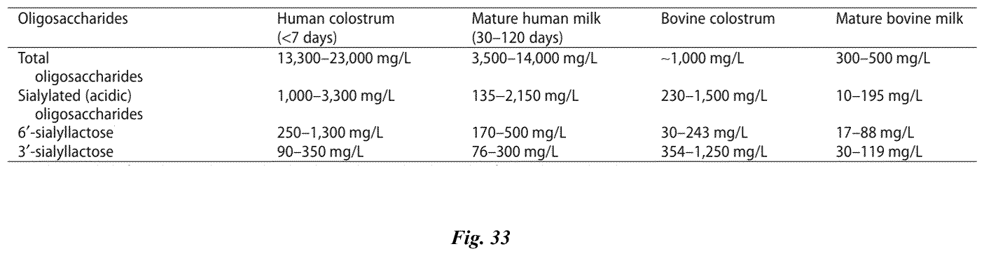

According to the present invention, the said cleaned edible bird's nest, ultra-low fat milk, purified water and optional ingredients are “synthesised” at a temperature above 90° C. for more than 10 minutes. According to the present invention, the “synthesising” process setting at below 90° C. is inefficient, where the conversion rate of protein-bound sialic acid to oligosaccharide-bound sialic acid is too low. FIG. 32 illustrated a detailed study of sialic acid concentration in the proposed nutritional product at different “synthesising” process temperature. Based on the study and data available in the present invention, it is concluded that the preferred “synthesising” process temperature and duration setting is between 110° C. and 130° C. for more than 10 minutes. The said preferred process setting is able to optimise release of said free and oligosaccharide-bound sialic acid from the said edible bird's nest. The said pressurised, heat-assisted hydrolysis process and the subsequent rapid cooling process are expected converting more than 15% of protein-bound sialic acid in the said edible bird's nest into said free and oligosaccharide-bound sialic acid, the preferable conversion rate is more than 50% of protein-bound sialic acid.

As illustrated in the FIG. 32, the said “synthesising” process is inefficient when the processing temperature is below 90° C., where the protein-bound sialic acid conversion rate is significantly below 10%. Meaning concentration of human colostrum likes oligosaccharide-bound sialic acid is below 10%, majority of the sialic acid is protein-bound. When the processing temperature is increased to above 110° C., observed a significant improvement in the concentration of free and oligosaccharide-bound sialic acids, as well as sialyllactose and sialyllactosamine and their derivatives or intermediates. Again, according to the present invention, the concentration of free and oligosaccharide-bound sialic acids, as well as sialyllactose and sialyllactosamine in the proposed nutritional product are best optimised when the “synthesising” process is at a temperature in the range of 110° C. to 130° C.

Besides, the said pressurised, heat-assisted hydrolysis process, and the subsequent rapid cooling process enhanced “synthesising” of sialyllactose and sialyllactosamine from said sialic acid in edible bird's nest, and said lactose/N-acetyllactosamine in milk. The said rapid cooling process is designed to rapidly cooling down the solution and promote more sialyllactose and sialyllactosamine formation. In this invention, the rapid cooling process is operated at a cooling rate of 5-95° C. per minute, the preferable cooling rate is at about 20° C. per minute.

Additionally, enzyme and/or mild acid are added as optional ingredients to further enhance or optimise the synthetisation of sialyllactose and sialyllactosamin. In fact, the formation of sialyllactose and sialyllactosamine are made possible by having another main ingredient during the processing, which is a mammalian milk, preferable an ultra-low fat milk with high concentration of lactose and N-acetyllactosamine.

In this invention, the milk fat composition of said mammalian milk is reduced to below 1.6% w/w, preferable the milk fat is reduced to between 0° C. and 0.1% w/w of the milk liquid. The said milk fat is removed via a centrifuging process or other milk fat removing process. According to this invention, the said ultra-low fat milk contains high concentration of lactose and N-acetyllactosamine (LacNAc), the combined lactose and N-acetyllactosamine content preferable more than 50% w/w of the milk solids, which is crucial to enable formation of sialyllactose and sialyllactosamine in the proposed nutritional product. In addition, the said ultra-low fat milk preferably comprising of sialyllactose concentrate derived from mammalian milk. The process for preparing said sialyllactose concentrate, comprising the steps of ultrafiltration of mammalian milk using an ultrafiltration membrane to obtain an ultrafiltration retentate. Then, diafiltration of the ultrafiltration retentate using an ultrafiltration membrane to get the mammalian milk's sialyllactose concentrate. The said ultrafiltration membrane has molecular weight cut off (MWCO) value from 0.5 to 5000 Dalton.

The said ultra-low fat mammalian milk, preferable a bovine or caprine milk. Bovine milk is a nutrient-dense food consisting of varying amounts of carbohydrate, fat, and protein. The major constituents of bovine milk are water (87.4%) and milk solids (12.6%), which includes vitamins, minerals, carbohydrate, fat, and protein. Bovine milk is a major component of non-breastfed infants' diets, used almost universally in infant formula as the most common human milk substitute. The principal carbohydrate in bovine milk is lactose. Fresh bovine milk contains about 4.8% lactose, whereas fresh human milk has 7% lactose.

A comparison of sialylated oligosaccharides, 3′-sialyllactose and 6′-sialyllactose in human milk and bovine milk is reported by Sandra J M ten Bruggencate as shown in FIG. 28. The comparison shown human colostrum contains 1000-3,300 mg/L of sialylated oligosaccharides, bovine colostrum contains 230-1,500 mg/L of sialylated oligosaccharides. Even though, bovine milk contains relatively lower amount of said oligosaccharide-bound sialic acid, sialyllactose, sialyllactosamine and N-acetylglucosamine than human milk, but bovine milk contains very high lactose. Therefore, bovine milk is consider a cost effective, natural and safe source of lactose and N-acetyllactosamine for the said nutritional product formulation according to the invention. Besides, sialyllactose concentrate can be derived from bovine milk via ultrafiltration and diafiltration processes. Hence, bovine milk is selected as one of the main raw ingredients in this invention.

Another objective of the invention is to provide a nutritional product with good sensory properties, which include appearance, texture, aroma and taste. According to this invention, an ultra-low fat milk with high lactose concentration is being used as one of the main ingredients. The ultra-low fat milk comprising of mammalian milk with less than 1.6% w/w of milk fat, preferable between 0° C. to 0.1% w/w of milk fat in the milk liquid to minimise lipid-bound sialic acid formation as well as to minimise chemical reaction between biomolecules of said milk fat and said edible bird's nest during “synthesising” process, which affect the sensory, physical, chemical and/or biological properties of the said nutritional product, where the said edible bird's nest will get harden and appear yellowish. In addition, when excessive chemical reaction happen between biomolecules of milk fat and edible bird's next, the sensory properties of the milk will be affected.

In order to formulate, synthesise and produce an effective nutritional product, the proposed plurality of processing methods are specifically designed and optimised to take advantage of high sialic acid and N-acetylglucosamine content in EBN, as well as high lactose and N-acetyllactosamine content in mammalian milk. There are a plurality of process combinations in implementing the present invention. FIG. 17 shown a summary of a plurality of process combinations according to the present invention. As illustrated in FIG. 17, the process combinations can be categorised into two main categories. The first process combinations category is the category which the “synthesising” of ingredients happen after the ingredients have been filled and sealed in individual containers (bottles, sachets or others). Four examples of the first process combinations are shown in FIG. 18, FIG. 19, FIG. 20 and FIG. 21. The second process combinations category is the category which the “synthesising” of ingredients happen before the ingredients being filled and sealed in individual containers (bottles, sachets or others). Six examples of the second process combinations are shown in FIG. 22, FIG. 23, FIG. 24, FIG. 25, FIG. 26 and FIG. 27.

Generally, the first process combinations category involved 7 main process steps as illustrated in FIG. 18. The process steps started with preparation of ingredients as per a pre-defined formulation, then followed by mixing of ingredients, homogenisation of ingredients, filling and sealing of ingredients in individual containers, “synthesising” of ingredients, pasteurising/sterilising of ingredients and final packaging. In the said first process combinations category, homogenisation of ingredients and pasteurising/sterilising of ingredients are the 2 optional process steps. According to the present invention, any one of the 2 optional process steps could be skipped for products which do not require to go thru the full list of process steps.

In addition, the second process combinations category involved 9 main process steps as illustrated in FIG. 22. The process step started with preparation of ingredients as per a pre-defined formulation, then followed by mixing of ingredients, homogenisation of ingredients, “synthesising” of ingredients, encapsulation of ingredients, pasteurising/sterilising of ingredient, spray drying/freeze drying of ingredients, filling and sealing of ingredients in individual containers and final packaging. In the second process combinations category, homogenisation of ingredients, encapsulation of ingredients, pasteurising/sterilising of ingredients, spray drying/freeze drying of ingredients are the 4 optional process steps. According to the present invention, any one of the 4 optional process steps could be skipped for products which do not require to go thru the full list of process steps.

According to present the invention, the process combinations are further illustrated by ten examples as shown in FIG. 18, FIG. 19, FIG. 20, FIG. 21, FIG. 22, FIG. 23, FIG. 24, FIG. 25, FIG. 26 and FIG. 27. As illustrated by FIG. 18, FIG. 19, FIG. 20, FIG. 21, FIG. 22, FIG. 23, FIG. 24, FIG. 25, FIG. 26 and FIG. 27, the four main ingredients categories of the present invention are edible bird's nest, mammalian milk, water and optional ingredients. The said edible bird's nest is a cleaned edible bird's nest. The said mammalian milk is preferable an ultra-low fat milk with high concentration of lactose and/or sialyllactose. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

In this invention, sugar and flavouring materials are added as optional ingredients for taste improvement. Enzyme and mild acid are added as another optional ingredients to further enhance or optimise the sialyllactose and sialyllactosamine synthetisation.

Besides, fucose and/or fucosyllactose are important optional ingredients to further perfect the formulation by mimicking all the 5 basic monosaccharides “building blocks” found in human milk oligosaccharide (HMO). The said fucosyllactose is an important fucosylated oligosaccharide, which could further enhance the nutritional value of the proposed nutritional product in improving not only the human brain health and immune function but the gut health too. In addition, sialyllactose and sialyllactosamine concentrate derived from mammalian milk are another optional ingredients added to further improve the formulation of said nutritional product.

In the present invention, the said plants extract included tocotrienols palm oil plants. The said omega-3 fatty acids included alpha-linolenic acid (ALA), eicosapentaenoic acid (EPA), and docosahexaenoic acid (DHA). The said probiotics included Lactobacilli spp., Eleutherine spp, and Bifidobacteria spp.

To date, many nutritional products did not demonstrate their full potential of health benefits due to poor absorption rate in human body. Larger particles size, low water solubility and low stability are mostly attributed to poor absorption in the human gastrointestinal (GI) tract. Orally administrated nutritional products must pass through the human GI tract and absorb by enteric epithelial cells. Therefore, it is another objective of the present invention to improve the absorption rate of the said nutritional product in the human gastrointestinal tract by introducing a homogenisation and encapsulation process in producing the said nutritional product, which is able to reduce ingredients' particle size, increase the solubility, stability and bioavailability of the said nutritional product.

According to the present invention, a new lipid-based encapsulation technique with high pressure homogenisation and ultra-sonication process is being proposed for entrapping a nutritional core material into a matrix of lipid coating material to improve the absorption rate of the said nutritional product in the human gastrointestinal tract, by reducing ingredients' particle size, increasing the solubility, stability and bioavailability of the said nutritional product.

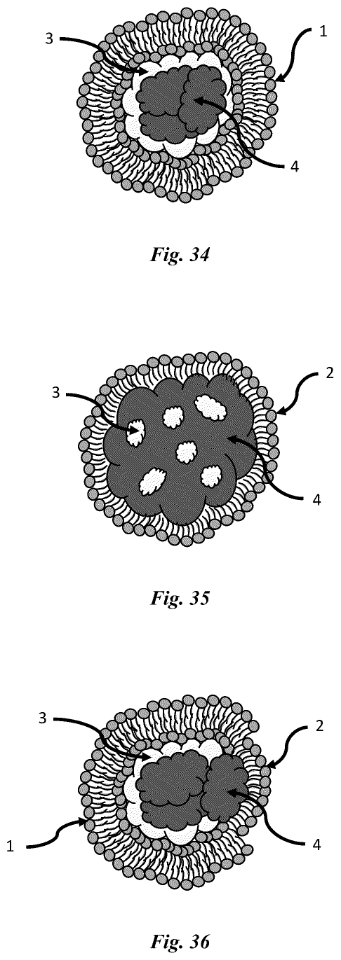

The proposed encapsulation technique involves encapsulating a micron-sized and/or nano-sized nutritional core material with a micron-sized and/or nano-sized thin layer of lipid coating. There are 3 types of lipid coating proposed in this invention. The said lipid coating included lipid bilayer (1), lipid monolayer (2) and the combination of both bilayer and monolayer, which enclosing the said nutritional core material as shown in FIG. 34, FIG. 35 and FIG. 36 respectively.

The first type of coating is lipid bilayer (1). This is when the said nutritional core materials is well surrounded with water, aqueous or hydrophilic components (3) of the ingredients, lipid bilayer (1) encapsulation will be formed perfectly as shown in FIG. 34. However, when most of the water, aqueous or hydrophilic components (3) of the ingredients are trapped inside the said nutritional core material, more hydrophobic components (4) of the ingredients are exposed at the surfaces of the said nutritional core material, more likely lipid monolayer (2) encapsulation will be formed as shown in FIG. 35.

In the third type of lipid coating proposed in this invention, where the said lipid coating involves a combination of both bilayer (1) and monolayer (2) enclosing on a single nutritional core material encapsulation, especially when the hydrophilic (3) & hydrophobic (4) components of the said nutritional core material is unevenly or imperfectly distributed as shown in FIG. 36. In this invention, the coating materials proposed in the said encapsulation coating layer are phospholipid, edible lipid and/or edible polymer.

According to the present invention, the said nutritional core material of the said encapsulation comprises of edible bird's nest, mammalian milk, water and optional ingredients, which were “synthesised” with the pre-defined setting. In fact, the said nutritional core material is the “formulated” and “synthesised” ingredients which undergo a series of processes, included mixing process and homogenisation process then followed by the “synthesising” process before subjected to the said encapsulation process as shown in FIG. 17.

During the encapsulation process the said core material, which is made of the said “synthesised” ingredients will be further homogenised to ensure an evenly distributed emulsion. Then, the said “synthesised” and “homogenised” ingredients are subjected to high-energy ultra-sonication process to encapsulate the nutritional core material with lipid layer.

In fact, during the said encapsulation process the said “formulated” and “synthesised” ingredients are further sizing down to micron-sized and/or nano-sized particles by a high-pressure homogeniser, high-shear homogeniser or other tools and then coating evenly with the said lipid coating layer. The diameter of the said micron-sized particles are ranging from 1-999 micrometres. The diameter of the said nano-sized particles are ranging from 1-999 nanometres.

FIG. 17 shown a summary flow chart of a plurality of process combinations according to the present invention. Finally, the plurality of methods to produce the said formulated and synthesised edible bird's nest and milk based nutritional product are further illustrated in examples 1, 2, 3, 4, 5, 6, 7, 8, 9 and 10 as shown in FIG. 18, FIG. 19, FIG. 20, FIG. 21, FIG. 22, FIG. 23, FIG. 24, FIG. 25, FIG. 26 and FIG. 27 respectively.

### EXAMPLE 1

According to example 1 of present invention as shown in FIG. 18, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a predefined weight ratio or quantity, the said ingredients included said cleaned edible bird's nest, said homogenised mammalian milk, said purified water and said optional ingredients;
f) homogenising all the said ingredients together;
g) filling and sealing the said ingredients in a plurality of containers;
h) synthesising the said ingredients, by a pressurised, heat-assisted hydrolysis and rapid cooling process;
i) pasteurising or sterilising the said ingredients;
j) final packaging.

According to example 1 of present invention as shown in FIG. 18. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactisamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to homogenise all the said ingredients together with a homogeniser, such as high pressure homogeniser.

The fourth step is to fill and seal the said ingredients in a plurality of containers, such as bottles and sachets.

The fifth step is to “synthesise” the said ingredients with a pressurised, heat-assisted hydrolysis and rapid cooling process.

The sixth step is to pasteurise or sterilise the said ingredients.

The seventh or final step is to do final packaging.

### EXAMPLE 2

According to example 2 of present invention as shown in FIG. 19, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a predefined weight ratio or quantity, the said ingredients included said cleaned edible bird's nest, said homogenised mammalian milk, said purified water and said optional ingredients;
f) filling and sealing the said ingredients in a plurality of containers;
g) synthesising the said ingredients, by a pressurised, heat-assisted hydrolysis and rapid cooling process;
h) pasteurising or sterilising the said ingredients together;
i) final packaging.

According to example 2 of present invention as shown in FIG. 19. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to fill and seal the said ingredients in a plurality of containers, such as bottles and sachets.

The fourth step is to “synthesise” the said ingredients with a pressurised, heat-assisted hydrolysis and rapid cooling process.

The fifth step is to pasteurise or sterilise the said ingredients.

The sixth or final step is to do final packaging.

### EXAMPLE 3

According to example 3 of present invention as shown in FIG. 20, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a predefined weight ratio or quantity, the said ingredients included said cleaned edible bird's nest, said homogenised mammalian milk, said purified water and said optional ingredients;
f) homogenising all the said ingredients together;
g) filling and sealing the said ingredients in a plurality of containers;
h) synthesising the said ingredients, by a pressurised, heat-assisted hydrolysis and rapid cooling process;
i) final packaging.

According to example 3 of present invention as shown in FIG. 20. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to homogenise all the said ingredients together with a homogeniser, such as high pressure homogeniser.

The fourth step is to fill and seal the said ingredients in a plurality of containers, such as bottles and sachets.

The fifth step is to “synthesise” the said ingredients with a pressurised, heat-assisted hydrolysis and rapid cooling process.

The sixth or final step is to do final packaging.

### EXAMPLE 4

According to example 4 of present invention as shown in FIG. 21, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a predefined weight ratio or quantity, the said ingredients included said cleaned edible bird's nest, said homogenised mammalian milk, said purified water and said optional ingredients;
f) filling and sealing the said ingredients in a plurality of containers;
g) synthesising the said ingredients, by a pressurised, heat-assisted hydrolysis and rapid cooling process;
h) final packaging.

According to example 4 of present invention as shown in FIG. 21. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to fill and seal the said ingredients in a plurality of containers, such as bottles and sachets.

The fourth step is to “synthesise” the said ingredients with a pressurised, heat-assisted hydrolysis and rapid cooling process.

The fifth or final step is to do final packaging.

### EXAMPLE 5

According to example 5 of present invention as shown in FIG. 22, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) synthesising the said ingredients, by a pressurised, heat-assisted hydrolysis and rapid cooling process;
h) encapsulating the said ingredients, by lipid bilayer (1) and/or lipid monolayer (2) coating as shown in FIG. 34, FIG. 35 and FIG. 36;
i) pasteurising or sterilising the said ingredients;
j) spray drying or freeze drying the said ingredients;
k) filling and sealing the said ingredients in a plurality of containers;
l) final packaging.

According to example 5 of present invention as shown in FIG. 22. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to homogenise all the said ingredients together with a homogeniser, such as high pressure homogeniser.

The fourth step is to “synthesise” the said ingredients with a pressurised, heat-assisted hydrolysis and rapid cooling process.

The fifth step is to encapsulate the said ingredients' particles, by lipid bilayer (1) and/or lipid monolayer (2) coating the micron-sized and/or nano-sized ingredients' particles.

The sixth step is to pasteurise or sterilise the said ingredients.

The seventh step is to spray dry/freeze dry the said ingredients;

The eighth step is to fill and seal the said ingredients in a plurality of containers, such as bottles and sachets.

The ninth or final step is to do final packaging.

### EXAMPLE 6

According to example 6 of present invention as shown in FIG. 23, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) synthesising the said ingredients, by a pressurised, heat-assisted hydrolysis and rapid cooling process;
h) encapsulating the said ingredients, by lipid bilayer (1) and/or lipid monolayer coating as shown in FIG. 34, FIG. 35 and FIG. 36;
i) pasteurising and sterilising the said ingredients;
j) filling and sealing the said ingredients in a plurality of containers;
k) final packaging.

According to example 6 of present invention as shown in FIG. 23. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to homogenise all the said ingredients together with a homogeniser, such as high pressure homogeniser.

The fourth step is to “synthesise” the said ingredients with a pressurised, heat-assisted hydrolysis and rapid cooling process.

The fifth step is to encapsulate the said ingredients' particles, by lipid bilayer (1) and/or lipid monolayer (2) coating the micron-sized and/or nano-sized ingredients' particles.

The sixth step is to pasteurise or sterilise the said ingredients.

The seventh step is to fill and seal the said ingredients in a plurality of containers, such as bottles and sachets.

The eighth or final step is to do final packaging.

### EXAMPLE 7

According to example 7 of present invention as shown in FIG. 24, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) synthesising the said ingredients, by a pressurised, heat-assisted hydrolysis and rapid cooling process;
h) encapsulating the said ingredients, by lipid bilayer (1) and/or lipid monolayer (2) coating as shown in FIG. 34, FIG. 35 and FIG. 36;
i) spray drying or freeze drying the said ingredients;
j) filling and sealing the said ingredients in a plurality of containers;
k) final packaging.

According to example 7 of present invention as shown in FIG. 24. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to homogenise all the said ingredients together with a homogeniser, such as high pressure homogeniser.

The fourth step is to “synthesise” the said ingredients with a pressurised, heat-assisted hydrolysis and rapid cooling process.

The fifth step is to encapsulate the said ingredients' particles, by lipid bilayer (1) and/or lipid monolayer (2) coating the micron-sized and/or nano-sized ingredients' particles.

The sixth step is to spray dry or freeze dry the said ingredients;

The seventh step is to fill and seal the said ingredients in a plurality of containers, such as bottles and sachets.

The eighth or final step is to do final packaging.

### EXAMPLE 8

According to example 8 of present invention as shown in FIG. 25, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) synthesising the said ingredients, by a pressurised, heat-assisted hydrolysis and rapid cooling process;
h) encapsulating the said ingredients, by lipid bilayer (1) and/or lipid monolayer (2) coating as shown in FIG. 34, FIG. 35 and FIG. 36;
i) filling and sealing the said ingredients in a plurality of containers;
j) final packaging.

According to example 8 of present invention as shown in FIG. 25. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to homogenise all the said ingredients together with a homogeniser, such as high pressure homogeniser.

The fourth step is to “synthesise” the said ingredients with a pressurised, heat-assisted hydrolysis and rapid cooling process.

The fifth step is to encapsulate the said ingredients' particles, by lipid bilayer (1) and/or lipid monolayer (2) coating the micron-sized and/or nano-sized ingredients' particles.

The sixth step is to fill and seal the said ingredients in a plurality of containers, such as bottles and sachets.

The seventh or final step is to do final packaging.

### EXAMPLE 9

According to example 9 of present invention as shown in FIG. 26, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) synthesising the said ingredients, by a pressurised, heat-assisted hydrolysis and rapid cooling process;
g) pasteurising/sterilising the said ingredients;
h) filling and sealing the said ingredients in a plurality of containers;
i) final packaging.

According to example 9 of present invention as shown in FIG. 26. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to “synthesise” the said ingredients with a pressurised, heat-assisted hydrolysis and rapid cooling process.

The fourth step is to pasteurise or sterilise the said ingredients.

The fifth step is to fill and seal the said ingredients in a plurality of containers, such as bottles and sachets.

The sixth or final step is to do final packaging.

### EXAMPLE 10

According to example 10 of present invention as shown in FIG. 27, the method of producing a formulated and synthesised edible bird's nest and milk based nutritional product with high concentration of oligosaccharide-bound sialic acid, N-acetylglucosamine, lactose and protein, comprising of the following steps:
a) preparing cleaned edible bird's nest, by cleaning raw edible bird's nest;
b) preparing homogenised mammalian milk, by homogenising mammalian milk;
c) preparing purified water, by purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) synthesising the said ingredients, by a pressurised, heat-assisted hydrolysis and rapid cooling process;
g) filling and sealing the said ingredients in a plurality of containers;
h) final packaging.

According to example 10 of present invention as shown in FIG. 27. The first step is to prepare all ingredients comprising of cleaned edible bird's nest, homogenised mammalian milk, purified water and optional ingredients. The said homogenised mammalian milk is preferable a homogenised ultra-low fat mammalian milk liquid with <1.6% milk fat. However, the said homogenised ultra-low fat mammalian milk could be substituted with a full cream mammalian milk or a sialyllactose concentrate added mammalian milk. The said optional ingredients included edible ingredients such as sugar, flavouring materials, enzyme, fucose, fucosyllactose, sialyllactose, sialyllactosamine, plants extract, fruits extract, garlic oil, essential oil, lipid, phospholipid, omega-3 fatty acids, folic acid, vitamin-C (ascorbic acid), vitamin-D, vitamin-E, probiotics, curcumin or other edible ingredients.

The second step is to mix all the said ingredients follow a pre-defined weight ratio as per the said nutritional product requirement or formulation. A plurality of examples of ingredients' weight ratio are illustrated in FIG. 38.

The third step is to “synthesise” the said ingredients with a pressurised, heat-assisted hydrolysis and rapid cooling process.

The fourth step is to fill and seal the said ingredients in a plurality of containers, such as bottles and sachets.

The fifth or final step is to do final packaging.

Although the invention has been described in detail for the purpose of illustration, it is understood that such detail is solely for that purpose. It is also to be noted that the present invention is susceptible to modifications, adaptations and changes by those skilled in the art. Such variant embodiments employing the concepts and features of this invention are intended to be within the scope of the present invention, which is further set forth under the following claims.

1. A formulated and synthesised, edible bird's nest and milk based nutritional product characterised by high concentration of oligosaccharide-bound sialic acid, N-acetylglucosamine, lactose and protein, comprising of edible bird's nest, mammalian milk, water and optional ingredients:
Wherein said nutritional product's formulation is mainly defined by the concentration of said oligosaccharide-bound sialic acid, said N-acetylglucosamine, said lactose and said protein in the said nutritional product;
Wherein said concentration of said oligosaccharide-bound sialic acid, said N-acetylglucosamine, said lactose and said protein in said nutritional product are more than 100 milligram/litre, 50 milligram/litre, 5 gram/litre and 5 gram/litre respectively; preferable concentration of said oligosaccharide-bound sialic acid, said N-acetylglucosamine, said lactose and said protein in said nutritional product are 3,300 milligram/litre, 1,600 milligram/litre, 50 gram/litre and 50 gram/litre respectively;
wherein said edible bird's nest comprising of cleaned edible bird's nest;
wherein said mammalian milk comprising of homogenised mammalian milk;
wherein said water comprising of purified water;
wherein said optional ingredients comprising of edible ingredients;
wherein the mixture of said edible bird's nest, said mammalian milk, said water and said optional ingredients are “synthesised” with a pressurised and heat-assisted hydrolysis process at temperature above 90° C. for more than 10 minutes, then follow by a rapid cooling process; preferable “synthesised” with a pressurised and heat-assisted hydrolysis process at a temperature in the range of 110° C. to 130° C. for more than 10 minutes, which able to optimise release of said free and oligosaccharide-bound sialic acid from the said edible bird's nest; the said pressurised and heat-assisted hydrolysis process and the said subsequent rapid cooling process converting more than 15% of protein-bound sialic acid in the said edible bird's nest into said free and oligosaccharide-bound sialic acid; preferable converting more than 50% of said protein-bound sialic acid in the said edible bird's nest into said free and oligosaccharide-bound sialic acid;
wherein weight ratio of said edible bird's nest to said mammalian milk liquid in the said nutritional product is ranging from 1:0.1 to 1:1000; preferable weight ratio of said edible bird's nest to said mammalian milk liquid in the said nutritional product is 1:25;
wherein weight ratio of said edible bird's nest to said water in the said nutritional product is ranging from 1:0.1 to 1:1000; preferable weight ratio of said edible bird's nest to said water in the said nutritional product is 1:4.

2. A formulated and synthesised, edible bird's nest and milk based nutritional product of claim 1, wherein said mammalian milk comprising of ultra-low fat mammalian milk with less than 1.6% w/w of milk fat, preferable between 0 to 0.1% w/w of milk fat in the said mammalian milk liquid.

3. A formulated and synthesised, edible bird's nest and milk based nutritional product of claim 1, wherein said mammalian milk comprising of mammalian milk with added sialyllactose concentrate derived from mammalian milk.

4. A formulated and synthesised, edible bird's nest and milk based nutritional product of claim 1, wherein the said pressurised and heat assisted hydrolysis process and the said subsequent rapid cooling process enhanced sialyllactose formation, an oligosaccharide formed from lactose and sialic acid.

5. A formulated and synthesised, edible bird's nest and milk based nutritional product of claim 1, wherein the said pressurised and heat assisted hydrolysis process and the said subsequent rapid cooling process enhanced sialyllactosamine formation, an oligosaccharide formed from N-acetyllactosamine (LacNAc) and sialic acid.

6. A formulated and synthesised, edible bird's nest and milk based nutritional product of claim 1, wherein enzyme and/or mild acid are added as optional ingredients to further enhance or optimise the synthetisation or formation of sialyllactose and sialyllactosamine.

7. A formulated and synthesised, edible bird's nest and milk based nutritional product of claim 1, wherein a fucose, fucolyllactose and/or their derivatives ingredients are added as optional ingredients to further improve the Human Milk Oligosaccharides (HMOs) contents in the said nutritional product.

8. A formulated and synthesised, edible bird's nest and milk based nutritional product of claim 1, wherein a sialyllactose concentrate derived from bovine and/or goat milk are added as optional ingredients to further improve the Human Milk Oligosaccharides (HMOs) contents in the said nutritional product.

9. A formulated and synthesised, edible bird's nest and milk based nutritional product of claim 1, wherein edible ingredients such as sugar, flavouring materials, enzyme, plants extract, fruits extract, garlic oil, essential oil, lipid, phospholipid, omega-3 fatty acids, folic acid, vitamin-C (ascorbic acid), vitamin-D, vitamin-E, probiotics, calcium, glucosamine and/or curcumin are added as optional ingredients.

10. A formulated and synthesised, edible bird's nest and milk based nutritional product of claim 1, wherein the said heat-assisted hydrolysis process and the said subsequent rapid cooling process is carried out in a pressurised environment with pressure above 0.10 MPa, the preferable pressure is in a range of 0.17 MPa to 0.19 MPa.

11. A formulated and synthesised, edible bird's nest and milk based nutritional product of claim 1, wherein the said ingredients of the said nutritional product are encapsulated with a lipid-based encapsulation technique, where the said ingredients are homogenised to become an evenly distributed emulsion, then the said micron-sized and/or nano-sized ingredients' particles are encapsulated with lipid bilayer (1) and/or lipid monolayer (2) coating as shown in FIG. 34, FIG. 35 and FIG. 36; the encapsulation process included subjected the said ingredients to high-energy sonication process.

12. A formulated and synthesised, edible bird's nest and milk based nutritional product of claim 1, wherein the said nutritional product is sterilised with a retort process, preferable at temperature above 105° C. for more than 10 minutes.

13. A formulated and synthesised, edible bird's nest and milk based nutritional product of claim 1, wherein the said nutritional product is sterilised with an Ultra-High Temperature (UHT) process, preferable at temperature above 135° C. for more than 2 seconds.

14. A formulated and synthesised, edible bird's nest and milk based nutritional product of claim 1, wherein the said nutritional product is pasteurised with a High Temperature Short Time (HTST) process, preferable at temperature above 71° C. for more than 15 seconds, then follow by rapid cooling.

15. A formulated and synthesised, edible bird's nest and milk based nutritional product of claim 1, wherein the said nutritional product is pasteurised with a High Heat Short Time (HHST) process, preferable at temperature above 89° C. for more than 1 seconds, then follow by rapid cooling.

16. A formulated and synthesised, edible bird's nest and milk based nutritional product of claim 1, wherein the said nutritional product is pasteurised with a Vat Pasteurisation process, preferable at temperature above 62° C. for more than 30 minutes, then follow by rapid cooling.

17. A formulated and synthesised, edible bird's nest and milk based nutritional product of claim 1, wherein said not hermetically sealed product is pasteurised with an Ultra Pasteurisation process, preferable at temperature above 137° C. for more than 2 seconds, then follow by rapid cooling.

18. A formulated and synthesised, edible bird's nest and milk based nutritional product of claim 1, wherein the said edible bird's nest source is originated from edible-nest swiftlets, included Aerodramus Fuciphagus and Aerodramus Maximus swiftlet species.

19. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) filling and then sealing all the said ingredients in a plurality of containers, such as bottles or sachets;
h) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
i) pasteurising or sterilising the said product; and
j) final packaging.

20. The nutritional product produced according to the method of claim 19.

21. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) filling and then sealing all the said ingredients in a plurality of containers, such as bottles or sachets;
g) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
h) pasteurising or sterilising the said product; and
i) final packaging.

22. The nutritional product produced according to the method of claim 21.

23. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) filling and then sealing all the said ingredients in a plurality of containers, such as bottles or sachets;
h) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
i) final packaging.

24. The nutritional product produced according to the method of claim 23.

25. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) filling and then sealing all the said ingredients in a plurality of containers, such as bottles or sachets;
g) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
h) final packaging.

26. The nutritional product produced according to the method of claim 25.

27. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
h) encapsulating the said ingredients, by a lipid-based encapsulation technique, where the said ingredients are further homogenised to ensure an evenly distributed emulsion, then encapsulate the said micron-sized and/or nano-sized ingredients' particle with a lipid bilayer (1) and/or lipid monolayer (2) coating as shown in FIG. 34, FIG. 35 and FIG. 36; the encapsulation process included subjected the said ingredients to high-energy sonication process;
i) pasteurising or sterilising the said ingredients;
j) spray drying or freeze drying the said ingredients;
k) filling and then sealing all the said ingredients in a plurality of containers, such as bottles or sachets; and
l) final packaging.

28. The nutritional product produced according to the method of claim 27.

29. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
h) encapsulating the said ingredients, by a lipid-based encapsulation technique, where the said ingredients are further homogenised to ensure an evenly distributed emulsion, then encapsulate the said micron-sized and/or nano-sized ingredients' particle with a lipid bilayer (1) and/or lipid monolayer (2) coating as shown in FIG. 34, FIG. 35 and FIG. 36; the encapsulation process included subjected the said ingredients to high-energy sonication process;
i) pasteurising or sterilising the said ingredients;
j) filling and then sealing all the said ingredients in a plurality of containers, such as bottles or sachets; and
k) final packaging.

30. The nutritional product produced according to the method of claim 29.

31. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
h) encapsulating the said ingredients, by a lipid-based encapsulation technique, where the said ingredients are further homogenised to ensure an evenly distributed emulsion, then encapsulate the said micron-sized and/or nano-sized ingredients' particle with a lipid bilayer (1) and/or lipid monolayer (2) coating as shown in FIG. 34, FIG. 35 and FIG. 36; the encapsulation process included subjected the said ingredients to high-energy sonication process;
i) spray drying or freeze drying the said ingredients;
j) filling and then sealing all the said ingredients in a plurality of containers, such as bottles or sachets; and
k) final packaging.

32. The nutritional product produced according to the method of claim 31.

33. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) homogenising all the said ingredients together;
g) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
h) encapsulating the said ingredients, by a lipid-based encapsulation technique, where the said ingredients are further homogenised to ensure an evenly distributed emulsion, then encapsulate the said micron-sized and/or nano-sized ingredients' particle with a lipid bilayer (1) and/or lipid monolayer (2) coating as shown in FIG. 34, FIG. 35 and FIG. 36; the encapsulation process included subjected the said ingredients to high-energy sonication process;
i) filling and then sealing all the said ingredients in a plurality of containers, such as bottles or sachets; and
j) final packaging.

34. The nutritional product produced according to the method of claim 33.

35. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
g) pasteurising or sterilising the said ingredients;
h) filling and then sealing all the said ingredients in a plurality of containers, such as bottles or sachets; and
i) final packaging.

36. The nutritional product produced according to the method of claim 35.

37. A method of producing a formulated and synthesised edible bird's nest and milk based nutritional product characterised by high concentration of oligosaccharide-bound sialic acid, N-acetylglucosamine, lactose and protein, comprising the steps of:
a) preparing cleaned edible bird's nest, by cleaning raw edible bird's nest;
b) preparing homogenised mammalian milk, by homogenising mammalian milk;
c) preparing purified water, by filtering and purifying municipal water;
d) preparing optional ingredients;
e) mixing all the said ingredients follow a pre-defined weight ratio or quantity, the said ingredients included said cleaned edible bird's nest, said homogenised mammalian milk, said purified water and said optional ingredients;
f) synthesising all the said ingredients, by a pressurised, heat-assisted hydrolysis and subsequent rapid cooling process; the said “synthesising” temperature is set at above 90° C. for more than 10 minutes; the preferable “synthesising” temperature is between 110° C. to 130° C. for more than 10 minutes to optimise the release of free sialic acid and formation of oligosaccharide-bond sialic acid from the said edible bird's nest and said mammalian milk; the synthetisation converting more than 15% of protein-bound sialic acid in the said edible bird's nest into said free and oligosaccharide-bound sialic acid; the said “synthesising” of ingredients also enhanced sialylated oligosaccharisdes formation, which included sialyllactose and sialyllactosamine.
g) filling and then sealing all the said ingredients in a plurality of containers, such as bottles or sachets; and
h) final packaging.

38. The nutritional product produced according to the method of claim 37.

39. A method of producing a formulated and synthesised, edible bird's nest and milk based nutritional product of claim 19, 21, 23, 25, 27, 29, 31, 33, 35 or 37, wherein said mammalian milk comprising of ultra-low fat mammalian milk with less than 1.6% w/w of milk fat, preferable between 0 to 0.1% w/w of milk fat in the said mammalian milk liquid.

40. A method of producing a formulated and synthesised, edible bird's nest and milk based nutritional product of claim 19, 21, 23, 25, 27, 29, 31, 33, 35 or 37, wherein said mammalian milk comprising of mammalian milk with added sialyllactose concentrate derived from mammalian milk.

41. A method of producing a formulated and synthesised, edible bird's nest and milk based nutritional product of claim 19, 21, 23, 25, 27, 29, 31, 33, 35 or 37, wherein the concentration of said oligosaccharide-bound sialic acid, said N-acetylglucosamine, said lactose and said protein in said nutritional product are more than 100 milligram/litre, 50 milligram/litre, 5 gram/litre and 5 gram/litre respectively; preferable concentration of said oligosaccharide-bound sialic acid, said N-acetylglucosamine, said lactose and said protein in said nutritional product are 3,300 milligram/litre, 1,600 milligram/litre, 50 gram/litre and 50 gram/litre respectively.

42. A method of increasing sialylated oligosaccharides contents in a lactating mother's human milk comprising of orally administering the said formulated and synthesised edible bird's nest and milk based nutritional product of claim 1, 20, 22, 24, 26, 28, 30, 32, 34, 36 or 38 to said lactating mother.

43. A method of increasing N-acetylglucosamine contents in a lactating mother's human milk comprising of orally administering the said formulated and synthesised edible bird's nest and milk based nutritional product of claim 1, 20, 22, 24, 26, 28, 30, 32, 34, 36 or 38 to said lactating mother.

44. A method of enhancing neurological development and brain health in a human comprising of orally administering the said formulated and synthesised edible bird's nest and milk based nutritional product of claim 1, 20, 22, 24, 26, 28, 30, 32, 34, 36 or 38 to said human.

45. A method of enhancing immune function in a human comprising orally administering the said formulated and synthesised edible bird's nest and milk based nutritional product of claim 1, 20, 22, 24, 26, 28, 30, 32, 34, 36 or 38 to said human.

46. A method of enhancing gut health in a human comprising orally administering the said formulated and synthesised edible bird's nest and milk based nutritional product of claim 1, 20, 22, 24, 26, 28, 30, 32, 34, 36 or 38 to said human.

47. A method of treating autism spectrum disorder (ASD) and/or seizures/epilepsy in a human comprising orally administering the said formulated and synthesised edible bird's nest and milk based nutritional product of claim 1, 20, 22, 24, 26, 28, 30, 32, 34, 36 or 38 to said human.

48. The said formulated and synthesised edible bird's nest and milk nutritional product according to claim 47, wherein Cannabidiol (CBD) and/or Tetrahydrocannabinoid (THC) are added as optional ingredients.
